(19) United States
(12) Patent Application Publication
(10) Pub. No.: US 2005/0159608 A1
(43) Pub. Date: Jul. 21, 2005

(54) Method of preparing an epoxidized functional vegetable oil

(76) Inventors: Robert B. Turner, Georgetown, TX (US); David M. Casper, Normal, IL (US)

Correspondence Address: MCKELLAR IP LAW, PLLC, 784 SOUTH POSEYVILLE ROAD, MIDLAND, MI, 48640, US

(21) Appl. No.: 11/028,275

(22) Filed: Jan. 3, 2005

Related U.S. Application Data
(60) Provisional application No. 60/537,827, filed on Jan. 20, 2004.

(51) Int. Cl. C07D 301/12

(52) U.S. Cl. 549/531

(57) ABSTRACT

A method of preparing an epoxide functional vegetable oil, the process comprises contacting a raw vegetable oil with hydrogen peroxide and an organic acid having from 1 to 20 carbon atoms, in the presence of water and a solvent for a sufficient period of time and at a controlled temperature of from 65° C. to 90° C. to form epoxide groups from unsaturated moieties in the vegetable oil.

### BACKGROUND OF THE INVENTION

The invention disclosed and claimed herein deals with a novel method of preparing epoxy functional vegetable oils and the vegetable oils so prepared.

Vegetable oils have been familiar to man since prehistoric times and for centuries, humans have used fats and oils for food and a variety of other uses. Humankind has over the years perfected the science surrounding the ability to produce oils from agriculture products for their own use. Today, millions of pounds of such oils are used in a variety of end use applications.

Vegetable oils are made up principally of triglycerides containing both saturated and unsaturated moieties, wherein the predominant moiety is the unsaturated variety. Eventhough the triglycerides lend themselves to many end used applications, there are some needs for these materials to have functionalities different than those that are found in the raw vegetable oil.

For example, the triglycerides can be converted to diol functional compounds, that is, multifunctional triglycerides, and the diol functional compounds can then be made useful, for example, in the formation of urethanes by reacting the diols with isocyanates. Coatings, elastomers, foams and composites can be made from elastomers using such diols. The triglycerides can also be converted to epoxide functional compounds and the epoxide functional compounds can then be used for forming other materials.

Currently, glycerides are hydrolyzed with water that is catalyzed by enzymes, acids, or metals to yield glycerol products, that is, where the ester groups are removed and replaced with carboxyl moities. Also, glycerides can be hydroformylated using rhodium catalysts or cobalt catalysts such as Co(Co)₆, and the polyaldehyde so-formed can be converted into polyol through hydrogenation with hydrogen and Raney nickel catalysts. Further, using oxidation, the polyaldhehydes can be converted into polycarboxylic acids. And finally, the polyaldhehydes can be converted to polyamines.

Fringuelli, and co-workers, have reported on a method to convert alkenes into 1,2-diols using peroxy acids in deionized water. The process involves the epoxidation of the alkene, and then the epoxide ring is directly opened by acid or base hydrolysis to produce the diol. It is stated by Fringuelli et al that the synthesis does not require organic solvents. (Friguelli, F., Germani, R, Pizzo, F. and Savelli, G., ONE-POT TWO-STEPS SYNTHESIS OF 1,2 DIOL, Synthetic Communications, 19(11 & 12), 1939-1943 (1989).

What has been discovered herein is a simple, economical preparative method for the provision of epoxy functional oils that are derived by converting the alkene groups of the unsaturated molecules that make up vegetable oils, into epoxide groups.

### BRIEF DESCRIPTION OF THE DRAWINGS

FIG. 1 is a schematic of the proposed reaction scheme to prepare the products of this invention.

### THE INVENTION

The invention described and claimed herein deals with a method of preparing epoxy functional vegetable oils. Thus, the invention comprises a method of preparing an epoxide functional vegetable oil, the process comprising contacting a raw vegetable oil with hydrogen peroxide and an organic acid having from 1 to 20 carbon atoms, in the presence of water and a solvent for a sufficient period of time and at a controlled temperature of from 65° C. to 90° C. to form epoxide groups from unsaturated moieties in the vegetable oil, and thereafter separating any volatiles from the epoxy functional vegetable oil at a temperature less than 90° C.

What is meant by “raw” vegetable oil is vegetable oil that has been obtained by normal processing techniques, without any modification to the chemistry of the oil itself. This vegetable oil can be crude or refined, and can be used as obtained from the producers.

The vegetable oil is contacted with hydrogen peroxide and an organic acid in the presence of water and a solvent. For purposes of this invention, the vegetable oil can be selected from any available vegetable oil, among which are the more common vegetable oils, such as corn oil, palm oil, soybean oil, cottonseed oil, peanut oil, rapeseed oil, safflower oil, and sunflower oil. Preferred for this invention are corn oil, cottonseed oil, and soybean oil, canola oil and most preferred are soybean oil and palm oil.

In the method, the vegetable oil is contacted with hydrogen peroxide and an organic acid in the presence of water and a solvent. The amount of hydrogen peroxide that is used ranges from about 0.7 to about 7.0 equivalents based on the amount of the amount of unsaturation that is in the vegetable oil. Larger amounts of the peroxide can be used, but there does not seem to be any desirable effect.

The amount of organic acid that is used is based on the amount of raw vegetable oil that is used, in that, there is used on the order of about 0.45 to about 2.0 molar equivalents in volume. For this invention, examples of organic acids that are useable are those having from 1 to 20 carbon atoms. Such acids are, for example, formic, acetic, propionic, n-butyric, isobutyric, 3-methylbutanoic, 2,2-dimethylpropanoic, n-valeric, n-caproic, n-heptoic, caprylic, n-nonylic, capric, undecylic, lauric, tridecylic, myristic, pentadecylic, palmitic, margaric, and stearic. Most preferred acids for this invention are formic and acetic because of the fact that they are essentially in liquid form at room temperature and are readily and economically available.

The amount of water that is used is based on the percent peroxide used and equivalents of raw vegetable oil. The amount of epoxide functionality in the vegetable oil is dependent on the amount of initial unsaturation found in the raw vegetable oil and the amount of peroxide that is used, as the amount of peroxide determines the amount of peracid that is formed, which in turn determines the amount of unsaturation that is converted into the epoxide. The peroxide that is used contains water.

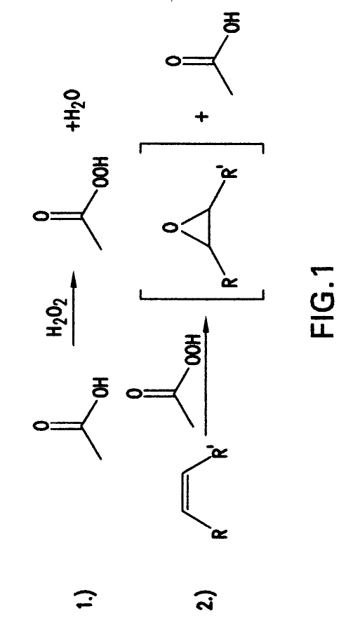

The inventor herein does not want to be held to such a theory, but the schematic reaction sequence illustrated in FIG. 1 demonstrates this point, wherein the designations R and R′ are the various segments forming the remainder of the vegetable oil. An organic acid, for example, acetic acid forms a peracid in the presence of peroxide, such as hydrogen peroxide, with formation of by-produced water, i.e. reaction scheme 1 (FIG. 1). Thereafter, the peracid reacts with the unsaturation in the vegetable oil to form the epoxide ring and a by-produced organic acid, as is shown in reaction scheme 2 (FIG. 1).

Solvents used in this method can be any aprotic solvent other than ethers.

For purposes of this invention, it has been found that the preferred solvents are hexanes, which includes all of the isomers of hexane. Hexane appears to enhance and control the reaction. Further, the process for the obtention of soybean oil requires that the soybean oil be extracted using hexane as the preferred solvent. It is contemplated within the scope of this invention to use such hexane solvated soybean oil and thus, eliminate the time consuming step of removal of the hexane before use as is now the industry process. Such a use, it can be observed saves both time and money.

The time that is required for this method ranges from about 1 hr. to about 24 hours. Normally, for the full conversion of the unsaturation to the epoxide configuration, the time required is about 1 to 4 hours.

The temperatures that are useful for the reactions can range from 66° C. to 90° and this temperature is controlled by the solvent and the type and/or mix of organic acids used, generally running at reflux temperatures.

It is preferred to add the peroxide and the organic acid to the vegetable oil at about the same time, but the method can be modified to first add the peroxide first and then the organic acid, or the organic acid can be added first and then the peroxide.

In the examples, 35% hydrogen peroxide was used as the source of peroxide, so a very large excess of water is present. An excess of peroxide is used in order to convert all of the alkenes to epoxides.

### EXAMPLES

Several samples of reaction mixtures were provided and the reactions run for the times and temperatures indicated in Table I. Hydrogen peroxide at 35% in water was used as the peroxide. There was used in each case, 75 gms. of crude soybean oil (1 equiv./24.404 mmole) having a molecular weight of 881, 19.84 ml of peroxide (9.5 equivalents, 231.84 mmole), 11.6 ml of Glacial Acetic Acid (0.475 m/v, 11.592 mmoles). The materials were heated slowly to the temperatures indicated and for the times indicated, which times were determined by the change of color of the reaction medium to white or clear, which is the indication of the conversion of the alkenes to the epoxides. The completed reactions were analyzed using FTIR. The results can be found in Table II

TABLE I
FORMULATIONS

| | Hexane | Hexane* | Toluene | Toluene | Temp./ | |
|---|---|---|---|---|---|---|
| Sample | Solvent | m/v eq | Solvent | m/v eq. | ° C. | Time |
| A | 6.1 ml | 0.25 | | | 89-90 | 4 hr. 17 min. |
| B | 12.2 ml | 0.50 | | | 76 | 23 hr. |
| C | 18.3 ml | 0.75 | | | 72 | 23 hr. |
| D | 24.2 ml | 1.00 | | | 69 | 23 hr. |
| E | | | 12.2 ml | 0.5 | 104 | 4 hr. 15 min. |

*m/v = molar volume

TABLE II

| Sample | Results | |
|---|---|---|
| A | 0% | crude soybean oil |
| | 84% | epoxidized soybean oil |
| B | 0% | crude soybean oil |
| | 78% | epoxidized soybean oil |
| C | 0% | crude soybean oil |
| | 86% | epoxidized soybean oil |
| | 14% | diol soybean oil |
| D | 0% | crude soybean oil |
| | 99.8% | epoxidized soybean oil |
| | 0.2% | diol soybean oil |
| E | 0% | epoxidized soybean oil |
| | 21.7% | diol soybean oil |
| | 72.3% | epoxidized soybean oil |

1. A method of preparing a multi-epoxidized vegetable oil, the process comprising contacting a raw vegetable oil with hydrogen peroxide and an organic acid having from 1 to 20 carbon atoms, in the presence of water and a solvent for a sufficient period of time and at a controlled temperature of from 65° C. to 90° C. to form epoxide groups from unsaturated moieties in the vegetable oil, and thereafter separating any volatiles from the epoxy functional vegetable oil at a temperature less than 90° C.

2. A method of preparing an epoxy functional vegetable oil as claimed in claim 1 wherein the vegetable oil is selected from the group consisting of corn oil, palm oil, soybean oil, cottonseed oil, peanut oil, rapeseed oil, safflower oil, and sunflower oil.

3. A method of preparing an epoxy functional vegetable oil as claimed in claim 1 wherein the raw vegetable oil is contacted with a combination of the peroxide and organic acid essentially simultaneously.

4. A method of preparing an epoxy functional vegetable oil as claimed in claim 1 wherein the raw vegetable oil is contacted with the organic acid first, and then contacted with the peroxide.

5. A method of preparing an epoxy functional vegetable oil as claimed in claim 1 wherein the raw vegetable oil is contacted with the peroxide first and then is contacted with the organic acid.

6. A method of preparing an epoxy functional vegetable oil as claimed in claim 1 wherein the time of reaction if from 1 to 24 hours.

7. A method of preparing an epoxy functional vegetable oil as claimed in claim 1 wherein the temperature ranges from about 66° C. to 72° C.

8. A method of preparing an epoxy functional vegetable oil as claimed in claim 1 wherein the amount of organic acid that is used ranges from about 0.4 molar equivalents in volume to about 2.0 molar equivalents in volume based on the amount of raw vegetable oil.

8. A method of preparing an epoxy functional vegetable oil as claimed in claim 1 wherein the organic acid that is used is a mixture of two or more organic acids.

10. A method of preparing an epoxy functional vegetable oil as claimed in claim 1 wherein the amount of peroxide that is used ranges from about 0.7 to about 7.0 equivalents based on the amount of unsaturation in the raw vegetable oil.

11. An epoxy functional vegetable oil when prepared by the method of claim 1.

12. A method of preparing an epoxy functional vegetable oil as claimed in claim 1 wherein the solvent is an aliphatic, aprotic solvent without the presence of ether solvents.

13. A method of preparing an epoxy functional vegetable oil as claimed in claim 12 wherein the solvent is hexane.
